(19) United States
(12) Patent Application Publication
(10) Pub. No.: US 2022/0400966 A1
(43) Pub. Date: Dec. 22, 2022

(54) BLOOD-PRESSURE MEASURING DEVICE AND METHOD FOR PRESENTING DATA OF BLOOD-PRESSURE MEASUREMENT

(71) Applicant: HEALTH & LIFE CO., LTD., New Taipei City (TW)

(72) Inventor: Meng-Yi LIN, New Taipei City (TW)

(21) Appl. No.: 17/834,714

(22) Filed: Jun. 7, 2022

(30) Foreign Application Priority Data
Jun. 15, 2021 (TW) 110121719

(51) Int. Cl. A61B 5/021; A61B 5/00; G16H 10/60

(52) U.S. Cl. CPC A61B 5/02116; A61B 5/743; G16H 10/60

(57) ABSTRACT

A method for presenting data of blood-pressure measurement includes: calculating blood-pressure index values based on entries of data of blood-pressure measurement; and presenting a blood-pressure trend interface that includes index level regions, and for each of the blood-pressure index values, an index level markers disposed in one of the index level regions. The blood-pressure trend interface is presented in a manner that the index level markers corresponding respectively to the blood-pressure index values cooperatively indicate a trend in the blood pressure.

### CROSS-REFERENCE TO RELATED APPLICATION

This application claims priority of Taiwanese Invention Patent Application No. 110121719, filed on Jun. 15, 2021.

### FIELD

The disclosure relates to a blood-pressure measuring device and a method for presenting data of blood-pressure measurement.

### BACKGROUND

A conventional blood-pressure measuring device usually only presents a single entry of data of blood-pressure measurement at one time, and thus it is inconvenient for a user to get a sense of the trend in the results of blood-pressure measurement over a time period.

### SUMMARY

Therefore, an object of the disclosure is to provide a blood-pressure measuring device and a method for presenting data of blood-pressure measurement that can alleviate at least one of the drawbacks of the prior art.

According to one aspect of the disclosure, the blood-pressure measuring device includes a storage, a display and a processor.

The storage is configured to store a plurality of entries of data of blood-pressure measurement in chronological order, and each of the plurality of entries of data of blood-pressure measurement corresponds to at least one result of blood-pressure measurement.

The processor is electrically connected to the storage and the display. The processor is configured to calculate a plurality of blood-pressure index values based on the plurality of entries of data of blood-pressure measurement, respectively, and to present a blood-pressure trend interface via the display. The blood-pressure trend interface includes a plurality of index level regions, and for each of the plurality of blood-pressure index values, an index level marker. The plurality of index level regions are associated respectively with a plurality of blood-pressure levels, are arranged in a first direction, and extend in parallel in a second direction that is perpendicular to the first direction. The index level marker is associated with the blood-pressure index value in one of the plurality of index level regions.

The blood-pressure trend interface is presented in a manner that the index level markers corresponding respectively to the plurality of blood-pressure index values cooperatively indicate a trend in blood pressure. The index level marker that corresponds to a greatest one of the plurality of blood-pressure index values is presented in one of the plurality of index level regions that is associated with a greatest one of the plurality of blood-pressure levels and that is arranged to be the last in the first direction. The index level marker that corresponds to a smallest one of the plurality of blood-pressure index values is presented in one of the plurality of index level regions that is associated with a smallest one of the plurality of blood-pressure levels and that is arranged to be the first in the first direction.

According to another aspect of the disclosure, the blood-pressure measuring method is to be implemented by the blood-pressure measuring device that is previously described, and includes steps of:

calculating a plurality of blood-pressure index values based on the plurality of entries of data of blood-pressure measurement, respectively; and

presenting a blood-pressure trend interface that includes
a plurality of index level regions which are associated respectively with a plurality of blood-pressure levels, which are arranged in a first direction, and which extend in parallel in a second direction that is perpendicular to the first direction, and
for each of the plurality of blood-pressure index values, an index level marker that is associated with the blood-pressure index value and that is disposed in one of the plurality of index level regions,

wherein the blood-pressure trend interface is presented in a manner that the index level markers corresponding respectively to the plurality of blood-pressure index values cooperatively indicate a trend in blood pressure, the index level marker that corresponds to a greatest one of the plurality of blood-pressure index values is presented in one of the plurality of index level regions that is associated with a greatest one of the plurality of blood-pressure levels and that arranged to be the last in the first direction, and the index level marker that corresponds to a smallest one of the plurality of blood-pressure index values is presented in one of the plurality of index level regions that is associated with a smallest one of the plurality of blood-pressure levels and that is arranged to be the first in the first direction.

### BRIEF DESCRIPTION OF THE DRAWINGS

Other features and advantages of the disclosure will become apparent in the following detailed description of the embodiment with reference to the accompanying drawings, of which:

FIG. 1 is a block diagram illustrating a blood-pressure measuring device according to an embodiment of the disclosure;

FIGS. 2 and 3 are flow charts cooperatively illustrating a method for presenting data of blood-pressure measurement according to an embodiment of the disclosure; and

FIG. 4 is a schematic diagram illustrating an example of a blood-pressure trend interface to be presented by the pressure measuring device that implements the method for presenting data of blood-pressure measurement.

### DETAILED DESCRIPTION

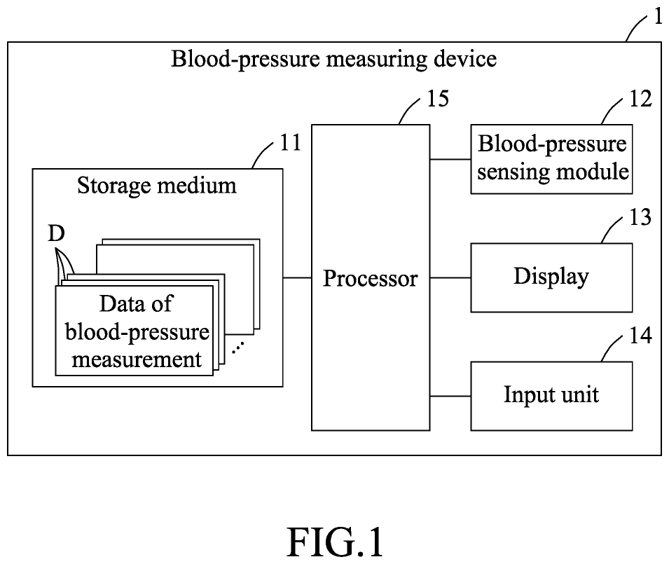

Referring to FIG. 1, an embodiment of a blood-pressure measuring device 1 according to the disclosure is illustrated. The blood-pressure measuring device 1 includes a storage medium 11, a blood-pressure sensing module 12, a display 13, an input unit 14, and a processor 15 that is electrically connected to the storage medium 11, the blood-pressure sensing module 12, the display 13 and the input unit 14.

The storage medium 11 may be implemented by random access memory (RAM), double data rate synchronous dynamic random access memory (DDR SDRAM), read only memory (ROM), programmable ROM (PROM), flash memory, a hard disk drive (HDD), a solid state disk (SSD), electrically-erasable programmable read-only memory (EEPROM) or any other volatile/non-volatile memory devices, but is not limited thereto. The storage medium 11 is configured to store a plurality of entries of data of blood-pressure measurement (D) in chronological order, and each of the entries of data of blood-pressure measurement (D) corresponds to one or more results of blood-pressure measurement that are generated during one time period. Specifically, the entries of data of blood-pressure measurement (D) correspond respectively to different time periods (e.g., different dates), and each result of blood-pressure measurement corresponding to one of the entries of data of blood-pressure measurement (D) is generated during the time period that corresponds to said one of the entries of data of blood-pressure measurement (D). Moreover, each of the entries of data of blood-pressure measurement (D) includes one or more values of systolic blood pressure and one or more values of diastolic blood pressure.

The blood-pressure sensing module 12 may be implemented by a cuff in combination with an inflatable air bladder and a pressure sensor, but is not limited thereto. The blood-pressure sensing module 12 is configured to be controlled by the processor 15 to implement an oscillometric method to perform blood-pressure measurement on a user in the separate time periods so as to generate the entries of data of blood-pressure measurement (D). Since implementation of the oscillometric method has been well known to one skilled in the relevant art, detailed explanation of the same is omitted herein for the sake of brevity.

The display 13 may be a set of seven-segment displays, a liquid-crystal display (LCD), a light-emitting diode (LED) display, a plasma display panel, a projection display or the like. The display 13 has either monochrome display capability or color display capability. However, implementation of the display 13 is not limited to the disclosure herein and may vary in other embodiments.

The input unit 14 may be implemented by a combination of multiple physical buttons, but is not limited thereto.

It is worth noting that, in some embodiments, the display 13 and the input unit 14 may be integrated as a touchscreen which has virtual buttons serving as the input unit 14.

The processor 15 may be implemented by a central processing unit (CPU), a microprocessor, a micro control unit (MCU), a system on a chip (SoC), or any circuit configurable/programmable in a software manner and/or hardware manner to implement functionalities discussed in this disclosure.

The processor 15 is configured to calculate, based on the entries of data of blood-pressure measurement (D), a plurality of blood-pressure index values that respectively correspond to the entries of data of blood-pressure measurement (D). It is worth noting that in some embodiments, each of the blood-pressure index values represents a mean arterial pressure (MAP), but is not limited thereto.

Based on the plurality of blood-pressure index values, the processor 15 is configured to generate a statistical result that includes a plurality of index level values which correspond respectively to the plurality of blood-pressure index values and each of which indicates one of a plurality of blood-pressure levels. In this embodiment, the plurality of blood-pressure levels exemplarily include “Level 1”, “Level 2”, “Level 3” and “Level 4” in ascending order, and each of the index level values may be one indicating the blood-pressure level of Level 1, two indicating the blood-pressure level of Level 2, three indicating the blood-pressure level of Level 3, or four indicating the blood-pressure level of Level 4.

Specifically, the processor 15 is configured to determine a greatest one and a smallest one of the plurality of blood-pressure index values, to calculate a level range based on the greatest one and the smallest one of the plurality of blood-pressure index values and a predetermined total number of the plurality of blood-pressure levels, and for each of the plurality of blood-pressure index values, to determine the index level value corresponding to the blood-pressure index value based on the level range. In this embodiment, the predetermined total number of the plurality of blood-pressure levels is four, but may be three, five or any positive integer greater than one in other embodiments.

More specifically, the processor 15 is configured to round the greatest one of the plurality of blood-pressure index values to obtain an integer as a rounded maximum, to round the smallest one of the plurality of blood-pressure index values to obtain an integer as a rounded minimum, and to calculate a difference between the rounded maximum and the rounded minimum. Then, the processor 15 is configured to divide the difference between the rounded maximum and the rounded minimum by the predetermined total number to obtain a first quotient as the level range.

Afterwards, for each of the plurality of blood-pressure index values, the processor 15 is configured to subtract the rounded minimum from the blood-pressure index value to obtain an index difference, to divide the index difference by the level range to obtain a second quotient, and to round down the second quotient to obtain a round number. Next, the processor 15 is configured to determine whether the round number is equal to the predetermined total number. The processor 15 is configured to make a sum of one and the round number serve as the index level value when it is determined that the round number is not equal to the predetermined total number, and to make the round number serve as the index level value when it is determined that the round number is equal to the predetermined total number.

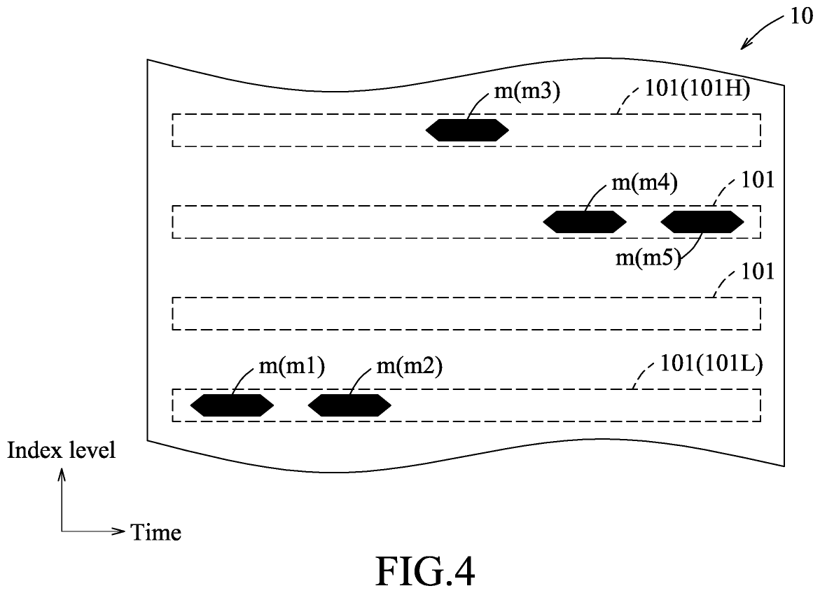

Subsequently, the processor 15 is configured to present a blood-pressure trend interface 10 (see FIG. 4) via the display 13.

The blood-pressure trend interface 10 includes a plurality of index level regions 101 which are associated respectively with the plurality of blood-pressure levels, which are arranged in a first direction, and which extend in parallel in a second direction that is perpendicular to the first direction. In the example of FIG. 4, the first direction is a bottom-to-top direction, and the second direction is a left-to-right direction.

The blood-pressure trend interface 10 further includes, for each of the plurality of blood-pressure index values, an index level marker (m) that is associated with the blood-pressure index value and that is disposed in one of the plurality of index level regions 101. The blood-pressure trend interface is presented in a manner that the index level markers (m) corresponding respectively to the plurality of blood-pressure index values cooperatively indicate a trend in blood pressure. The higher the position of an index level marker (m) in the blood-pressure trend interface 10, the higher the corresponding blood-pressure level (which is positively related to the corresponding blood-pressure index value), and the more to the right the position of an index level marker (m) in the blood-pressure trend interface 10, the later the time period that corresponds to the entry of data of blood-pressure measurement (D) to which the corresponding blood-pressure index value corresponds.

Specifically, the processor 15 is configured to, for each of the plurality of blood-pressure index values, present the index level marker (m) that corresponds to the blood-pressure index value in one of the plurality of index level regions 101 that is associated with one of the plurality of blood-pressure levels indicated by the index level value which corresponds to the blood-pressure index value.

In particular, in the blood-pressure trend interface 10 generated by the processor 15, the index level marker (m) that corresponds to the greatest one of the plurality of blood-pressure index values is presented in one of the plurality of index level regions 101 that is associated with a greatest one of the plurality of blood-pressure levels and that is arranged to be the last in the first direction (hereinafter referred to as “the highest index level region (101H)”). The index level marker (m) that corresponds to the smallest one of the plurality of blood-pressure index values is presented in another one of the plurality of index level regions 101 that is associated with a smallest one of the plurality of blood-pressure levels and that is arranged to be the first in the first direction (hereinafter referred to as “the lowest index level region (101L)”).

It is worth noting that whenever the greatest one and the smallest one of the plurality of blood-pressure index values are unequal, the index level markers (m) that respectively correspond to the greatest one and the smallest one of the plurality of blood-pressure index values would be presented in two different ones of the plurality of index level regions 110 (i.e., the highest index level region 101H and the lowest index level region 101L), and thus the trend in the blood pressure could be clearly indicated in blood-pressure trend interface 10 presented by the blood-pressure measuring device 1 according to the disclosure.

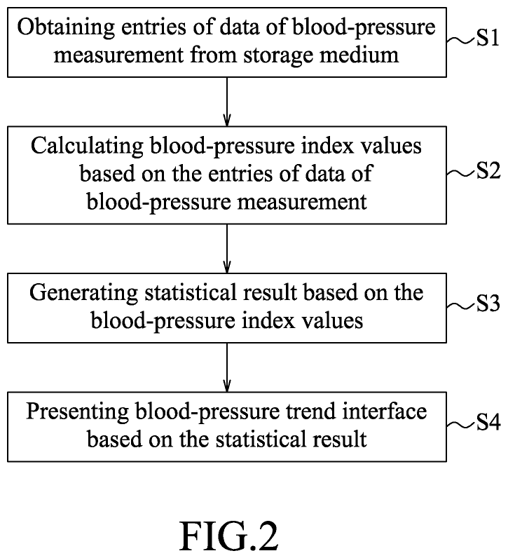

Referring to FIG. 2, an embodiment of a method for presenting data of blood-pressure measurement according to the disclosure is illustrated. The method is to be implemented by the blood-pressure measuring device 1 that is previously described, and includes steps S1 to S4 delineated below.

In step S1, in response to receipt of a command that is generated based on a user operation of the input unit 14 and that indicates multiple times of blood-pressure measurement performed during certain time periods, the processor 15 of the blood-pressure measuring device 1 obtains from the storage medium 11 a plurality of entries of data of blood-pressure measurement (D) that respectively correspond to the certain time periods. For example, the certain time periods are five consecutive days from Mar. 7, 2021 to Mar. 11, 2021 (i.e., each time period is one day), and the processor 15 obtains five entries of data of blood-pressure measurement (D) respectively corresponding to the five consecutive days. It should be noted that the certain time periods may be nonconsecutive days.

In step S2, the processor 15 calculates a plurality of blood-pressure index values based on the entries of data of blood-pressure measurement (D), respectively.

Specifically, for each of the entries of data of blood-pressure measurement (D), the processor 15 divides a sum of the one or more values of systolic blood pressure included in the entry of data of blood-pressure measurement (D) by a total number of the one or more values of systolic blood pressure to obtain a first arithmetic mean (i.e., an arithmetic mean of the one or more values of systolic blood pressure), and divides a sum of the one or more values of diastolic blood pressure included in the entry of data of blood-pressure measurement (D) by a total number of the one or more values of diastolic blood pressure to obtain a second arithmetic mean (i.e., an arithmetic mean of the one or more values of diastolic blood pressure). Next, the processor 15 multiplies the first arithmetic mean by one third to obtain a first weighted value, multiplies the second arithmetic mean by two third to obtain a second weighted value, and makes a sum of the first weighted value and the second weighted value serve as the blood-pressure index value.

In step S3, based on the plurality of blood-pressure index values, the processor 15 generates a statistical result that includes a plurality of index level values which correspond respectively to the plurality of blood-pressure index values and each of which indicates one of a plurality of blood-pressure levels in one of the certain time periods.

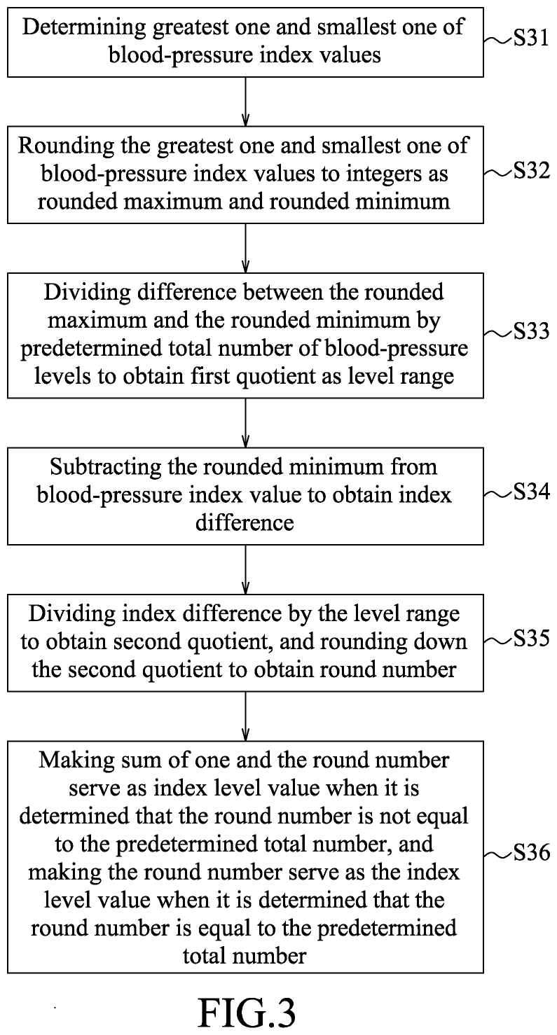

Referring to FIG. 3, the step of generating a statistical result includes sub-steps S31 to S36 delineated below.

In sub-step S31, the processor 15 determines a greatest one and a smallest one of the plurality of blood-pressure index values. In an example where five blood-pressure index values that respectively correspond to the five entries of data of blood-pressure measurement (D) and that are calculated in step S2 are “83.7”, “84.0”, “89.7”, “87.7” and “88.1” in chronological order, the processor 15 determines “89.7” and “83.7” respectively as the greatest one and the smallest one of the five blood-pressure index values.

In sub-step S32, the processor 15 rounds the greatest one of the plurality of blood-pressure index values to obtain an integer as a rounded maximum, and rounds the smallest one of the plurality of blood-pressure index values to obtain an integer as a rounded minimum. For example, the processor 15 rounds up the greatest one of the five blood-pressure index values “89.7” to be the rounded maximum “90”, and rounds down the smallest one of the five blood-pressure index values “83.7” to be the rounded minimum “83”. It should be noted that the way of rounding is not limited to the disclosure herein and may vary in other embodiments.

In sub-step S33, the processor 15 calculates a difference between the rounded maximum and the rounded minimum, and divides the difference between the rounded maximum and the rounded minimum by the predetermined total number to obtain a first quotient as the level range. For example, the processor 15 calculates a difference between the rounded maximum “90” and the rounded minimum “83” as “7”, and divides the difference “7” by the predetermined total number “4” to obtain a first quotient “1.75” as the level range.

For each of the plurality of blood-pressure index values, sub-steps S34 to S36 in the following are executed to obtain the index level value corresponding to the blood-pressure index value.

In sub-step S34, the processor 15 subtracts the rounded minimum from the blood-pressure index value to obtain an index difference.

In sub-step S35, the processor 15 divides the index difference by the level range to obtain a second quotient, and rounds down the second quotient to obtain a round number.

In sub-step S36, the processor 15 determines whether the round number is equal to the predetermined total number. The processor 15 makes a sum of one and the round number serve as the index level value when it is determined that the round number is not equal to the predetermined total number, and makes the round number serve as the index level value when it is determined that the round number is equal to the predetermined total number.

For example, the rounded minimum “83” is subtracted from each of the five blood-pressure index values “83.7”, “84.0”, “89.7”, “87.7” and “88.1” to result in five index differences “0.7”, “1.0”, “6.7”, “4.7” and “5.1”, respectively (sub-step S34). Following that, each of the five index differences “0.7”, “1.0”, “6.7”, “4.7” and “5.1” is divided by the level range “1.75” and then the quotient is rounded down to five round numbers “0”, “0”, “3”, “2” and “2”, respectively (sub-step S35). Since each of the five round numbers is not equal to the predetermined total number “4”, the processor 15 adds one to each of the five round numbers to result in five index level values “1”, “1”, “4”, “3” and “3”, respectively (sub-step S36). The statistical result generated in this example is summarized in Table 1 below.

TABLE 1

| Blood- | | | | | Index |
|---|---|---|---|---|---|
| pressure | Rounded | Level | Index | Round | level |
| index value | maximum/minimum | range | difference | number | value |
| 83.7 | 83 | 1.75 | 0.7 | 0 | 1 |
| (smallest) | | | | | |
| 84.0 | | | 1.0 | 0 | 1 |
| 89.7 | 90 | | 6.7 | 3 | 4 |
| (greatest) | | | | | |
| 87.7 | | | 4.7 | 2 | 3 |
| 88.1 | | | 5.1 | 2 | 3 |

In step S4, the processor 15 presents the blood-pressure trend interface 10 via the display 13 based on the statistical result generated in step S3. For each of the plurality of blood-pressure index values, the processor 15 presents the index level marker (m) that corresponds to the blood-pressure index value in one of the plurality of index level regions 101 that is associated with one of the plurality of blood-pressure levels indicated by the index level value which corresponds to the blood-pressure index value.

For example, referring to FIG. 4, four index level regions 101 from bottom to up are associated respectively with the four blood-pressure levels “Level 1”, “Level 2”, “Level 3” and “Level 4”, and five index level markers (m1, m2, m3, m4 and m5) from left to right respectively correspond to the five blood-pressure index values “83.7”, “84.0”, “89.7”, “87.7” and “88.1”. Because the five blood-pressure index values “83.7”, “84.0”, “89.7”, “87.7” and “88.1” respectively correspond to the five index level values “1”, “4”, “3” and “3” each indicating one of the four blood-pressure levels “Level 1”, “Level 2”, “Level 3” and “Level 4”, the index level markers (m1, m2) are presented in a first one of the four index level regions 101 (i.e., the lowest index level region (101L)) that is associated with the blood-pressure level “Level 1”, the index level marker (m3) is presented in a fourth one of the four index level regions 101 (i.e., the highest index level region (101H)) that is associated with the blood-pressure level “Level 4”, and the index level markers (m4, m5) are presented in a third one of the four index level regions 101 that is associated with the blood-pressure level “Level 3”.

To sum up, the blood-pressure measuring device 1 and the method for presenting data of blood-pressure measurement according to the disclosure provide the blood-pressure trend interface 10 in a manner that each of the index level markers (m) is presented in the corresponding one of the index level regions 101, facilitating a user to observe a trend in results of blood-pressure measurement in an intuitive way. Accordingly, the user can detect abnormality of the blood pressure at an early stage. Moreover, the way of presenting the blood-pressure trend interface 10 according to the disclosure can be realized on a display that only has limited display capabilities. Even if the display 13 of the blood-pressure measuring device 1 is implemented by a set of seven-segment displays, segments “A”, “G” and “D” of each of the set of seven-segment displays could be utilized to represent the index level markers (m).

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A method for presenting data of blood-pressure measurement, to be implemented by a blood-pressure measuring device, the blood-pressure measuring device storing a plurality of entries of data of blood-pressure measurement in chronological order, each of the plurality of entries of data of blood-pressure measurement corresponding to at least one result of blood-pressure measurement, the method comprising steps of:
calculating a plurality of blood-pressure index values based on the plurality of entries of data of blood-pressure measurement, respectively; and
presenting a blood-pressure trend interface that includes
a plurality of index level regions which are associated respectively with a plurality of blood-pressure levels, which are arranged in a first direction, and which extend in parallel in a second direction that is perpendicular to the first direction, and
for each of the plurality of blood-pressure index values, an index level marker that is associated with the blood-pressure index value and that is disposed in one of the plurality of index level regions,
wherein the blood-pressure trend interface is presented in a manner that the index level markers corresponding respectively to the plurality of blood-pressure index values cooperatively indicate a trend in blood pressure, the index level marker that corresponds to a greatest one of the plurality of blood-pressure index values is presented in one of the plurality of index level regions that is associated with a greatest one of the plurality of blood-pressure levels and that is arranged to be the last in the first direction, and the index level marker that corresponds to a smallest one of the plurality of blood-pressure index values is presented in one of the plurality of index level regions that is associated with a smallest one of the plurality of blood-pressure levels and that is arranged to be the first in the first direction.

2. The method as claimed in claim 1, before the step of presenting a blood-pressure trend interface, further comprising steps of:
based on the plurality of blood-pressure index values, generating a statistical result that includes a plurality of index level values which correspond respectively to the plurality of blood-pressure index values and each of which indicates one of the plurality of blood-pressure levels,
wherein the step of presenting a blood-pressure trend interface includes for each of the plurality of blood-pressure index values, presenting the index level marker that corresponds to the blood-pressure index value in one of the plurality of index level regions based on the index level value corresponding to the blood-pressure index value.

3. The method as claimed in claim 2, wherein the step of generating a statistical result includes sub-steps of:
determining the greatest one and the smallest one of the plurality of blood-pressure index values;
calculating a level range based on the greatest one and the smallest one of the plurality of blood-pressure index values and a predetermined total number of the plurality of blood-pressure levels; and
for each of the plurality of blood-pressure index values, determining the index level value corresponding to the blood-pressure index value based on the level range.

4. The method as claimed in claim 3, wherein the sub-step of calculating a level range includes sub-steps of:
rounding the greatest one of the plurality of blood-pressure index values to obtain an integer as a rounded maximum;
rounding the smallest one of the plurality of blood-pressure index values to obtain an integer as a rounded minimum;
calculating a difference between the rounded maximum and the rounded minimum; and
dividing the difference between the rounded maximum and the rounded minimum by the predetermined total number to obtain a first quotient as the level range.

5. The method as claimed in claim 4, wherein for each of the plurality of blood-pressure index values, the sub-step of determining the index level value includes sub-steps of:
subtracting the rounded minimum from the blood-pressure index value to obtain an index difference;
dividing the index difference by the level range to obtain a second quotient;
rounding down the second quotient to obtain a round number;
determining whether the round number is equal to the predetermined total number;
making a sum of one and the round number serve as the index level value when it is determined that the round number is not equal to the predetermined total number; and
making the round number serve as the index level value when it is determined that the round number is equal to the predetermined total number.

6. The method as claimed in claim 2, wherein the step of presenting a blood-pressure trend interface includes for each of the plurality of blood-pressure index values, presenting the index level marker that corresponds to the blood-pressure index value in one of the plurality of index level regions that is associated with one of the plurality of blood-pressure levels indicated by the index level value which corresponds to the blood-pressure index value.

7. A blood-pressure measuring device, comprising:
a storage medium configured to store a plurality of entries of data of blood-pressure measurement, each of the plurality of entries of data of blood-pressure measurement corresponding to at least one result of blood-pressure measurement;
a display; and
a processor electrically connected to said storage medium and said display, and configured to
calculate a plurality of blood-pressure index values based on the plurality of entries of data of blood-pressure measurement, respectively, and
present via said display a blood-pressure trend interface that includes
a plurality of index level regions which are associated respectively with a plurality of blood-pressure levels, which are arranged in a first direction, and which extend in parallel in a second direction that is perpendicular to the first direction, and
for each of the plurality of blood-pressure index values, an index level marker that is associated with the blood-pressure index value and that is disposed in one of the plurality of index level regions,
wherein the blood-pressure trend interface is presented in a manner that the index level markers corresponding respectively to the plurality of blood-pressure index values cooperatively indicate a trend in blood pressure, the index level marker that corresponds to a greatest one of the plurality of blood-pressure index values is presented in one of the plurality of index level regions that is associated with a greatest one of the plurality of blood-pressure levels and that is arranged to be the last in the first direction, and the index level marker that corresponds to a smallest one of the plurality of blood-pressure index values is presented in one of the plurality of index level regions that is associated with a smallest one of the plurality of blood-pressure levels and that is arranged to be the first in the first direction.

8. The blood-pressure measuring device as claimed in claim 7, wherein:
before presenting the blood-pressure trend interface, said processor is further configured to, based on the plurality of blood-pressure index values, generate a statistical result that includes a plurality of index level values which correspond respectively to the plurality of blood-pressure index values and each of which indicates one of the plurality of blood-pressure levels; and
said processor is configured to, for each of the plurality of blood-pressure index values, present the index level marker that corresponds to the blood-pressure index value in one of the plurality of index level regions based on the index level value corresponding to the blood-pressure index value.

9. The blood-pressure measuring device as claimed in claim 8, wherein said processor is configured to generate the statistical result by:
determining the greatest one and the smallest one of the plurality of blood-pressure index values;
calculating a level range based on the greatest one and the smallest one of the plurality of blood-pressure index values and a predetermined total number of the plurality of blood-pressure levels; and
for each of the plurality of blood-pressure index values, determining the index level value corresponding to the blood-pressure index value based on the level range.

10. The blood-pressure measuring device as claimed in claim 9, wherein said processor is configured to calculate the level range by:
rounding the greatest one of the plurality of blood-pressure index values to obtain an integer as a rounded maximum;
rounding the smallest one of the plurality of blood-pressure index values to obtain an integer as a rounded minimum;
calculating a difference between the rounded maximum and the rounded minimum; and
dividing the difference between the rounded maximum and the rounded minimum by the predetermined total number to obtain a first quotient as the level range.

11. The blood-pressure measuring device as claimed in claim 10, wherein for each of the plurality of blood-pressure index values, said processor is configured to determine the index level value by:
subtracting the rounded minimum from the blood-pressure index value to obtain an index difference;
dividing the index difference by the level range to obtain a second quotient;
rounding down the second quotient to obtain a round number;
determining whether the round number is equal to the predetermined total number;
making a sum of one and the round number serve as the index level value when it is determined that the round number is not equal to the predetermined total number; and
making the round number serve as the index level value when it is determined that the round number is equal to the predetermined total number.

12. The blood-pressure measuring device as claimed in claim 8, wherein said processor is configured to, for each of the plurality of blood-pressure index values, present the index level marker that corresponds to the blood-pressure index value in one of the plurality of index level regions that is associated with one of the plurality of blood-pressure levels indicated by the index level value which corresponds to the blood-pressure index value.
